(19) United States
(12) Patent Application Publication
(10) Pub. No.: US 2026/0351371 A1
(43) Pub. Date: Nov. 5, 2026

(54) MACHINE-LEARNING HEALTHCARE ASSISTANCE TOOL WITH A WEARABLE COMPUTING DEVICE

(71) Applicant: OpticSurg Inc., Newark, DE (US)

(72) Inventor: Tran Tu Huynh, New York, NY (US)

(73) Assignee: OpticSurg Inc., Newark, DE (US)

(21) Appl. No.: 19/664,495

(22) Filed: Apr. 30, 2026

Related U.S. Application Data
(60) Provisional application No. 63/797,853, filed on Apr. 30, 2025.

(51) Int. Cl. G16H 20/00; G16H 40/20; G16H 80/00

(52) U.S. Cl. CPC G16H 20/00; G16H 40/20; G16H 80/00

(57) ABSTRACT

Systems, methods, and devices disclosed herein include an healthcare training tool implemented using a wearable computing device. The system includes one or more processors and one or more memory devices storing instructions which, when executed by the one or more processors, cause the system to perform multiple operations to assist in training a healthcare personnel and/or generate and present a healthcare personnel assessment. The operations include determining, based on input data generated by the wearable computing device, a treatment scenario indicator corresponding to the healthcare treatment encounter. The treatment scenario indicator and at least a portion of the input data are provided to a trained machine-learning (ML) model as model inputs with instructions to calculate a statistical likelihood value corresponding to a patient outcome based on the model inputs. An indication of an ML-based output from the trained ML model is presented at the wearable computing device.

### CROSS-REFERENCE TO RELATED APPLICATION

This application claims priority to U.S. Provisional Patent Application Ser. No. 63/797,853, filed Apr. 30, 2025, and titled “MACHINE-LEARNING HEALTHCARE ASSISTANCE TOOL WITH A WEARABLE COMPUTING DEVICE,” the entirety of which is incorporated by reference herein.

### BACKGROUND

### 1. Field

This disclosure relates to healthcare devices and, more particularly, wearable device technology for healthcare personnel training and/or decision support.

### 2. Discussion of Related Art

The healthcare industry is experiencing a shortage of frontline care clinicians, staff, and skilled workers, straining the resources for training new employees. Typically, a frontline clinicians, staff, and skilled workers can work under the supervision of a more experienced employee for up to six years to receive training when they join a new facility, work environment, or specialty. This requires significant time from the more experienced employees during which the experienced employees may be limited in performing their other duties. The training period can be extensive due to the wide variety of treatment scenarios in which the experienced employee's input is needed. In addition, when newly on their own, clinicians, staff, and skilled workers may also need to consult and seek advice and guidance from more experienced personnel. Depending on the rare nature of special occurrences, even experienced clinicians, staff, and skilled workers may need to consult and seek advice and guidance from more specialized personnels.

### SUMMARY

Systems, methods, and devices disclosed herein can address the aforementioned issues. In some instances, a healthcare training and/or decision support system includes a wearable computing device, one or more processors, and/or one or more memory devices storing instructions which, when executed by the one or more processors, cause the system to perform various operations. The operations can include generating, at the wearable computing device, input data including audio data and imagery data representing a healthcare treatment encounter with a patient. The system can also determine, based on the input data, a treatment scenario indicator corresponding to the healthcare treatment encounter. Additionally, the system can provide the treatment scenario indicator and/or at least a portion of the input data to a trained machine-learning (ML) model as model inputs. The trained ML model can be instructed to calculate a statistical likelihood value corresponding to a patient outcome based on the model inputs. The system can receive an ML-based output from the trained ML model responsive to the instructing of the trained ML model to calculate the statistical likelihood value. Moreover, the system can present an indication of the ML-based output at the wearable computing device.

In some examples, the determining of the treatment scenario indicator can include providing the imagery data to an image recognition model as a first model input, receiving a predicted environment data output from the image recognition model, providing the audio data to a audio-to-text converter model as a second model input, receiving a text data output from the audio-to-text converter model, and/or providing the predicted environment data output and the text data output to a scenario prediction model as third model inputs. The treatment scenario indicator can include one or more treatment context factors, such as at least one of a treatment facility type a treatment facility unit or a determination of a rural treatment region or an urban treatment region. Additionally, the treatment scenario indicator can includes one or more patient factors including at least one of an injury type, an injury severity, one or more patient-provided symptoms, one or more vital signs, one or more patient test results, or one or more patient risk factors. The treatment scenario indicator can also include one or more healthcare personnel factors. The healthcare personnel factors can include at least one of a healthcare personnel occupation, a healthcare personnel specialty, a healthcare personnel experience level, or a prior treatment activity.

In some scenarios, the instructions, when executed by the one or more processors, can cause the system to instruct the trained ML model to determine one or more treatment actions that increase the statistical likelihood value corresponding to the patient outcome. The instructions can also cause the system to receive a treatment action output from the trained ML model and/or present an indication of the treatment action output at a display of the wearable computing device. Furthermore, the instructions, when executed by the one or more processors, can cause the system to provide a training data set to an ML model resulting in the trained ML model. The training data set can include a plurality of previous healthcare encounters associated with training labels. Moreover, the training labels can include context factor labels, patient factor labels, healthcare personnel factor labels, treatment action labels, and/or patient outcome labels.

In some instances, the ML model can be a previously-trained global ML model provided at a server of a cloud service, and/or the plurality of previous healthcare encounters can correspond to a particular healthcare facility implementing an ML-based healthcare assistance platform from the cloud service. In this way, the trained ML model can be a localized trained ML model corresponding to the particular healthcare facility. Additionally, the input data can be first input data, the ML-based output can be a first ML-based output, and the instructions, when executed by the one or more processors, can cause the system to provide second input data representing a plurality of healthcare treatment encounters, particular to a wearer of the wearable computing device, to the trained ML model. The instructions can also cause the system to receive, from the trained ML model responsive to the second input data, a second ML-based output representing a healthcare personnel assessment, and/or cause an indication of the second ML-based output to be presented at the wearable computing device. Moreover, the healthcare personnel assessment can include a value calculated to represent a readiness level for a particular procedure.

In some examples, a healthcare training and/or decision support system can include one or more processors and one or more memory devices storing instructions which, when executed by the one or more processors, cause the system to perform one or more operations. The operations can include receiving input data generated by a wearable computing device, the input data including audio data and imagery data representing a healthcare treatment encounter with a patient. The system can also determine, based on the input data, a treatment scenario indicator corresponding to the healthcare treatment encounter and/or provide the treatment scenario indicator and/or at least a portion of the input data to a trained ML model as model inputs. The operations can additionally include instructing the trained ML model to calculate a statistical likelihood value corresponding to a patient outcome based on the model inputs. Then, the system can receive a first ML-based output from the trained ML model responsive to the instructing of the trained ML model to calculate the statistical likelihood value. Moreover, the system can instruct the trained ML model to determine one or more treatment actions that increase the statistical likelihood value corresponding to the patient outcome. The system can receive a second ML-based output from the trained ML model responsive to the instruction of the trained ML model to determine the one or more treatment actions. Furthermore, the system can cause an indication of the second ML-based output to be presented at the wearable computing device.

In some scenarios, the patient outcome can be selected, based at least in part on the treatment scenario indicator, from a group of patient outcomes including one or more of a likelihood of complications, a likelihood of regaining mobility, a likelihood of returning to work within a predetermined time frame, a likelihood of requiring an operation, or a likelihood of infection. The treatment scenario indicator can include a treatment context factor, a patient factor, and a healthcare personnel factor. The causing of the indication of the second ML-based output to be presented at the wearable computing device can include causing a visualization of the one or more treatment actions to be overlayed on an a live-feed of a real-world image of the healthcare treatment encounter.

In some instances, a method of healthcare treatment assistance with a wearable computing device can include receiving input data generated by the wearable computing device representing a healthcare treatment encounter with a patient and/or determining, based on the input data, a treatment scenario indicator corresponding to the healthcare treatment encounter. The method can also include providing the treatment scenario indicator and at least a portion of the input data to a trained ML model as model inputs, providing a first instruction the trained ML model to calculate a plurality of statistical likelihood value corresponding to a patient outcome based on the model inputs, and/or receiving a first ML-based output from the trained ML model responsive to the first instruction. Additionally, the method can include causing an indication of the first ML-based output to be presented at the wearable computing device.

In some examples, the method can include providing a second instruction to the trained ML model to determine one or more treatment actions that increase the statistical likelihood value corresponding to the patient outcome, receiving a second ML-based output from the trained ML model responsive to the instruction of the trained ML model to determine the one or more treatment actions, and/or causing an indication of the second ML-based output to be presented at the wearable computing device. The input data can include audio data and imagery data. Also, the determining of the treatment scenario indicator can include providing the imagery data to an image recognition model as a first model input, receiving a predicted environment data output from the image recognition model, providing the audio data to a audio-to-text converter model as a second model input, receiving a text data output from the audio-to-text converter model, and/or providing the predicted environment data output and the text data output to a scenario prediction model as third model inputs.

In some scenarios, the method can include establishing a wireless network connection between the wearable computing device and a local clinician device associated with a treatment facility of a the person wearing the wearable computing device. The trained ML model can be stored at the local clinician device. Additionally, the method can include training a global ML model, at a cloud service device, with a first training data set resulting in a trained global ML model at the cloud service device, sending the global ML model to the local clinician device resulting in a local ML model, and/or training the local ML model with a second training data set, particularized for the treatment facility, resulting in the trained ML model as a localized trained ML model.

### BRIEF DESCRIPTION OF THE FIGURES

FIG. 1A illustrates an example system for implementing a healthcare assistance platform on a wearable computing device.

FIG. 1B illustrates an example system for implementing a healthcare assistance platform on a wearable computing device using a trained machine-learning (ML) model.

FIG. 2 illustrates an example system for implementing a healthcare assistance platform on a wearable computing device with a model training procedure.

FIG. 3 illustrates an example system for implementing a healthcare assistance platform on a wearable computing device using a localized ML model.

FIG. 4 illustrates an example system for implementing a healthcare assistance platform using one or more computing device(s) and/or a wireless network architecture.

FIG. 5 illustrates an example method of implementing a healthcare assistance platform, which can be performed by any of the systems of FIGS. 1A-4.

### DETAILED DESCRIPTION

It will be appreciated that numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

Systems, methods, and devices disclosed herein can include a healthcare training and/or assistance system implemented using a wearable computing device. Input data for the healthcare assistance system can be generated by the wearable computing device. After data pre-processing, a first machine-learning (ML) model can determine treatment scenario indicators corresponding to the healthcare treatment encounter. The treatment scenario indicators and/or at least a portion of the input data from the wearable computing device can be provided to a second ML model as model inputs with instructions to calculate a statistical likelihood value corresponding to a patient outcome based on these model inputs. An indication of an ML-based output from the second ML model can be presented at the wearable computing device and can include one or more patient outcomes, recommended treatment actions for increasing a statistical likelihood of the patient outcomes, and/or an assessment of a readiness or competency level of the personnel using the assistance/training system (e.g., for a particular task and/or an overall position performance level).

In some instances, the disclosed system(s) can include a localized ML model particular to a single healthcare treatment facility or group of treatment facilities operated by a particular entity. The localized ML model can continually be retaught based on new information, facility results, and/or changing policies of the particular entity using this technology. Furthermore, a validation system can determine consistencies and difference between recommendations outputted by the localized ML model and those actions actually selected to be taken by the user. The validation system can also integrate with quality metrics of the particular hospital (e.g., documentation on number of days until discharge) and/or industry metrics and standards. In some scenarios, using industry guidelines as model inputs, such as National Comprehensive Cancer Network (NCCN) guidelines for cancer treatment, can decrease the learning burden and/or can make the recommendations more efficient. The system(s) can also use the CPT codes of each diagnosis to organize and/or validate the localized training data, further improving efficiency. Moreover, the system(s) disclosed herein can help the user learn to discern more minor or benign injuries/illnesses from those that are more serious and require urgent attention, such as identifying a difference between inflammation and infection, and can make a corresponding recommendation.

Accordingly, the disclosed technology can create advantages by determining a flow for how care is given particularized to different healthcare treatment scenarios. By receiving model inputs, such as training data, from wearable devices, the training data can have significantly increased variety. The smaller, lighter form factor of the wearable device enables it to travel with the user throughout nearly every stage of the healthcare treatment encounters, providing a rich abundance of data that can be categorized into a variety of unique treatment scenarios. For example, the treatment scenarios can be determined from input data drawn from bedside care, the intensive care unit, the hospital floor, experiences in the operating room, office encounters, or any other location throughout the healthcare treatment facility.

Additional advantages of the systems, methods, and devices discussed herein will become apparent from the detailed description below.

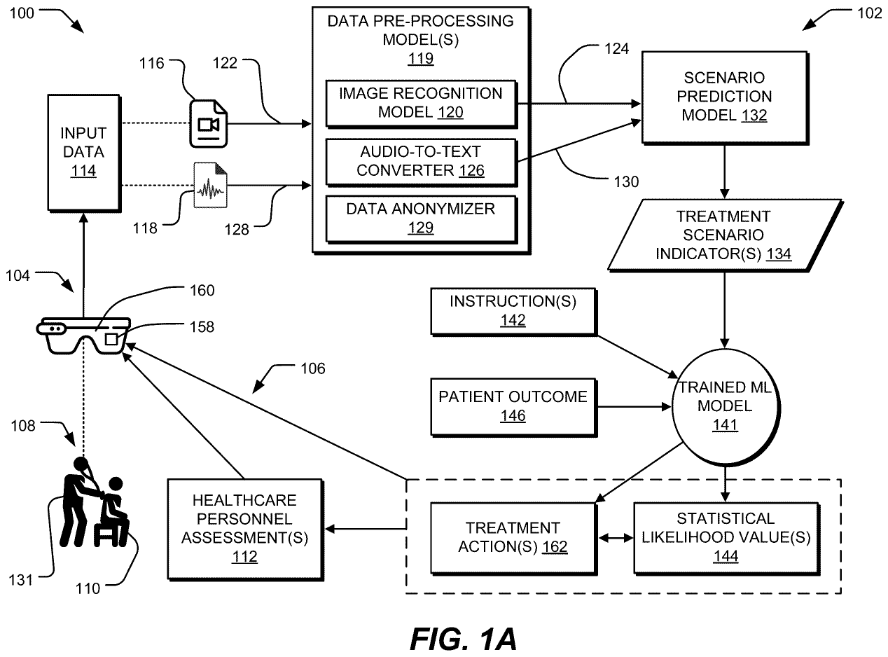

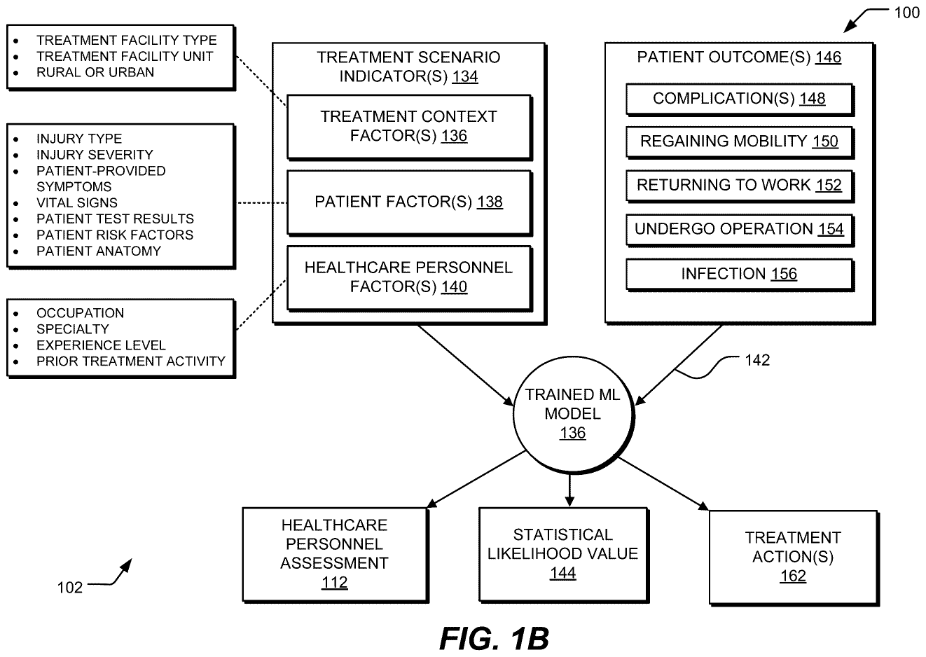

FIGS. 1A and 1B illustrate an example system 100 for implementing a healthcare assistance platform 102 on a wearable computing device 104. The system 100 depicted in FIGS. 1A and 1B can be used to provide real-time, ML-assisted training feedback 106 during a healthcare treatment encounter 108 with a patient 110, The system(s) 100 can also provide one or more healthcare personnel assessment(s) 112, as discussed in greater detail below.

In some examples, as shown in FIG. 1A, the wearable computing device 104 (e.g., smart glasses) can generate the input data 114 representing the healthcare treatment encounter 108. For instance, the input data 114 can include imagery data 116 (e.g., video data) generated by one or more sensors of the wearable computing device 104 (e.g., camaras, light sensors, motion sensors, infrared sensors, or so forth). The input data 114 can also include audio data 118 generated by the one or more sensors the wearable computing device 104 (e.g., acoustic sensors, microphones, or so forth).

In some scenarios, the input data 114 can be sent to one or more data pre-processing models 119. The imagery data 116 can be sent to an image recognition model 120 as first model input data 122. The image recognition model 120 can generate a predicted environment data output 124 based on the imagery data 116. For example, the predicted environment data output 124 can indicate a predicted environment in which the healthcare treatment encounter 108 is occurring based on the objects detected by the image recognition model 120. The predicted environment can include one or more of an emergency room (ER), a procedure room, a nursing home, a minor emergency clinic, an at-home care, an ambulance and/or first responder vehicle, a scene of an accident, or so forth. Furthermore, the audio data 118 can be sent to an audio-to-text converter 126 as second model input data 128. The audio-to-text converter 126 can generate a text data output 130 representing sounds and/or speaking occurring at the healthcare treatment encounter 108, such as by a wearer 131 of the wearable computing device 104 and/or the patient 110. Additionally, a data anonymizer 129 can remove personally identifiable information (e.g., a name, a facial image, a tattoo, a scar, a birth mark, an age, a voice sound, or so forth) from the imagery data 116, the audio data 118, the predicted environment data output 124, and/or the text data output 130, resulting in anonymized input data, which can be sent to a scenario prediction model 132.

In some cases, the scenario prediction model 132 can determine one or more treatment scenario indicator(s) 134 based on the model inputs provided to the scenario prediction model 132 (e.g., the predicted environment data output 124, and/or the text data output 130 from the data pre-processing models 119). The treatment scenario indicator(s) 134 can include characterizations of the particular features of the healthcare treatment encounter 108. The scenario prediction model 132 can perform various supervised and unsupervised learning operations to match the treatment scenario indicator(s) 134 to previously determined labels from training, and/or to discover patterns from the input data indicative of the treatment scenario indicator(s) 134.

For instance, as shown in FIG. 1B, the treatment scenario indicator(s) 134 can include one or more treatment context factor(s) 136, one or more patient factor(s) 138, one or more healthcare personnel factor(s) 140, and/or any combination thereof. The treatment context factor(s) 136 can include a treatment facility type (e.g., emergency room, minor emergency clinic, hospital, primary care clinic, pediatric clinic, specialist clinic, an ambulance/first responder vehicle, a field clinic, or so forth), a treatment facility unit (e.g., intensive care unit, emergency medicine, general surgery, radiology, neonatal intensive care unit, oncology, burn, anesthesiology, gastroenterology, outpatient, ambulatory services, orthopedics, elderly services, cardiology, hematology, pediatrics, labor and delivery, gynecology, psychiatry, coronary care, operating room, physical medicine/rehabilitation, neurology, and/or combinations thereof), and/or a determination of whether the treatment facility is in a rural region or an urban region. Additionally, the patient factor(s) 138 can include an injury type, an injury severity, one or more patient-provided symptoms, one or more vital signs, one or more patient test results, one or more patient risk factors (e.g., diet, smoking, height-to-weight ratio, housed vs unhoused, blood pressure, or so forth), and/or patient anatomy data (e.g., height, weight, age, gender, etc.). The patient factor(s) 138 can further sub-divide into injury/illness factors and patient factors independent of the injury/illness (e.g., patient risk factors, patient anatomy). Finally, the healthcare personnel factor(s) 140 can include data related to the healthcare personnel providing the treatment (e.g., the wearer 131 of the wearable computing device 104) and can include an occupation, a specialty, an experience level, a prior treatment activity (e.g., provided at a previous healthcare treatment encounter), and/or any combination thereof. These hierarchical categories and sub-divisions, disclosed herein, can result in the improved efficiency of the data storage hierarchy, as well as improved results from the trained ML model 136.

In some examples, the scenario prediction model 132 can determine these treatment scenario indicator(s) 134 from the predicted environment data output 124, and/or the text data output 130, and can send the treatment scenario indicator(s) 134 to a trained machine learning (ML) model 141 as ML model inputs 138. The imagery data 116, the audio data 118, the predicted environment data output 124, and/or the text data output 130 can be provided to the trained ML model 141 in addition to (e.g., or alternatively to) providing the treatment scenario indicator(s) 134. Furthermore, one or more instruction(s) 142 can be provided to the trained ML model 141 to generate one or more outputs based on the ML model inputs 138.

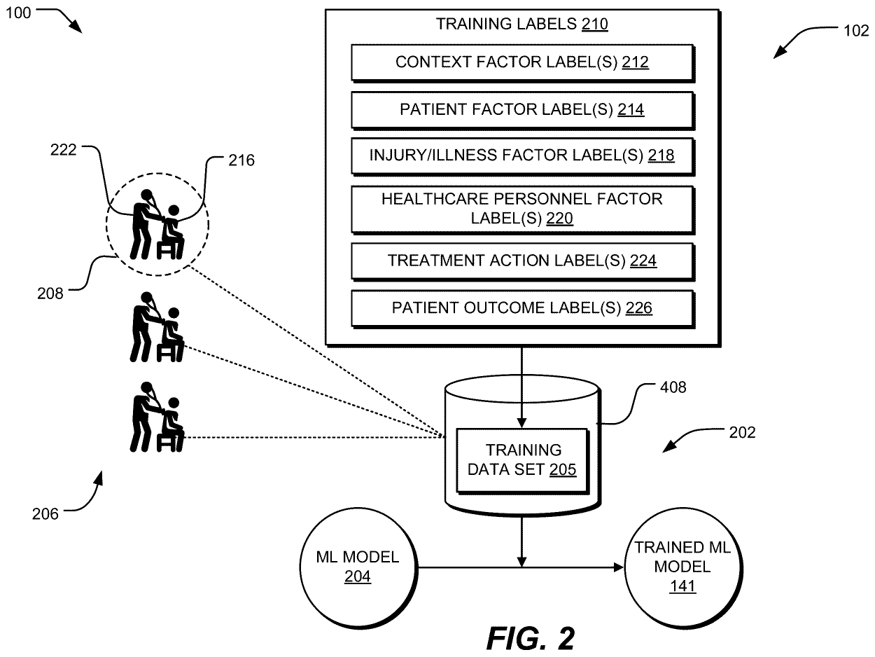

For instance, the instruction(s) 142 can include an instruction to calculate one or more statistical likelihood value(s) 144 corresponding to one or more patient outcome(s) 146 based on the ML model inputs 138. The instruction(s) 142 can identify one or more particular patient outcome(s) 146 for which statistical likelihood value(s) 144 are to be calculated. Additionally or alternatively, the instruction 142 can provoke the trained ML model 141 to identify the patient outcome(s) 146 (e.g., based on the ML training discussed below regarding FIG. 2). As shown in FIG. 1B, the patient outcome(s) 146 can include one or more complication(s) 148, regaining mobility 150, returning to work 152, undergoing an operation 154, getting an infection 156, and/or any combination thereof. The trained ML model 141 can determine if any of the patient outcome(s) fall below one or more statistical likelihood threshold values. In response to the instruction(s) 142, the trained ML model 141 can output the statistical likelihood value(s) 144 and/or a comparison to the threshold value(s).

Furthermore, the patient outcome(s) 146 can be associated with a particular patient treatment and/or outcome timeline. For instance, the a healthcare assistance platform 102 can establish a clinical outcome pathway (e.g., timeline) for the patient, including expected dates (e.g., or ranges of dates) for certain treatment actions and/or expected outcomes to occur. In these scenarios, the predicted patient outcome(s) 146 can be associated with a particular phase or moment in time of the timeline (e.g., a date, a week, a month, etc.). In response to determining the predicted patient outcome(s) 146 and/or its relation to the clinical outcome pathway, the clinical outcome pathway (e.g., and/or expected treatment actions or predictions of the clinical outcome pathway) can be adjusted to account for the predicted patient outcome(s) 146. Moreover, additionally or alternatively to using the patient outcome(s) 146, the clinical outcome pathway (e.g., and/or expected treatment actions or predictions of the clinical outcome pathway) can be continually and/or periodically adjusted based on real-time, real-world data of the patient received by the healthcare assistance platform 102.

In some cases, an indication 158 of the statistical likelihood value(s) 144 can be presented at the wearable computing device 104, for example, as a visual representation overlayed onto real-time imagery of the healthcare treatment encounter 108 at a display 160 of the wearable computing device 104 (e.g., as an augmented reality (AR) indicator). The indication 158 can also include an auditory output at the wearable computing device 104 using one or more speakers of the wearable computing device 104, and/or the indication 158 can include haptic feedback generated at the wearable computing device 104. By way of example, the indication 158 can include a combination of all three of visual, auditory, and haptic indicators.

In some scenarios, the instruction(s) 142 can include an instruction 142 to determine one or more treatment action(s) 162 which can increase the statistical likelihood value 144. This instruction 142 can be sent responsive to a determination that the initial statistical likelihood value 144 is below the predetermined likelihood threshold value. Furthermore, the trained ML model 141 can determine the treatment action(s) 162 which will cause the statistical likelihood value 144 to be greater than the predetermined likelihood threshold value. The treatment action(s) 162 can be outputted by the trained ML model 141 and presented at the wearable computing device 104 in addition to and/or alternatively to the statistical likelihood value 144 itself. The treatment action(s) 162 can include one or more of prescribing a particular medication, a particular medication dosage, a change to a medication dosage, an additional question to ask during the healthcare treatment encounter 108, a blood test, an imaging procedure, a physical therapy instruction, a particular examination action, a second opinion, a patient monitoring instruction or duration, and/or any combination thereof. These treatment action(s) 162 can be provided as one or more recommendations generated by a recommendation engine for reaching a predetermined likelihood threshold value for a particular statistical likelihood value 144 of the patient outcome 146.

For example, the recommendation engine can generate the recommendation based on the provided patient outcome 146 and/or a patient outcome 146 determined by the trained ML 141 itself, in some cases, based on patterns detected of patient outcomes achieved in the previous healthcare treatment encounters 208. In one use-case, the recommendation engine can use one or more treatment scenario indicator(s) 134 (e.g., patient factors) indicating a falling accident as an injury type, knee pain as a patient provided symptoms, and between ages 65-70 as a patient risk factor. The healthcare personnel factor(s) 140 can be an occupation of any of a nurse, a doctor, a surgeon an anesthesiologist, a surgical scrub tech, a circulating nurse, an intensive care unit nurse, and so forth. The healthcare personnel factor 140 can include a data entry indicating whether the position of the healthcare personnel is a sterile position or a non-sterile position and/or how the sterile status can change during the treatment flow associated with the treatment scenario indicators 134. Additionally, the recommendation can be organized based on CPT codes or other identifiers used by the healthcare treatment facility.

Furthermore, in some instances, the instruction(s) 142 can include an instruction to determine the healthcare personnel assessment 112. For instance, the ML model inputs 138 can include a plurality of treatment scenario indicator(s) 134 corresponding to a plurality of healthcare treatment encounters 108 for a particular healthcare personnel (e.g., the wearer 131 of the wearable computing device 104). The trained ML model 141 can assess a level of readiness and/or a level of competency for the particular healthcare personnel by determining a plurality of statistical likelihood value(s) 144 for the patient outcome(s) 146, and/or by comparing the plurality of statistical likelihood value(s) 144 to predetermined likelihood threshold values. Additionally, the trained ML model 141 can assess patient outcome data associated with the plurality of healthcare treatment encounters 108 to determine the healthcare personnel assessment 112. The healthcare personnel assessment 112 can include a rating or other calculated value representing the readiness and/or competency level of the particular healthcare personnel. Moreover, the healthcare personnel assessment 112 can be presented at the wearable computing device 104 and/or at another computing device, such as a healthcare treatment facility computing device (e.g., associated with a supervisor of the particular healthcare personnel).

FIG. 2 illustrates an example system 100 for implementing the healthcare assistance platform 102 on a wearable computing device 104. As depicted in FIG. 2, the healthcare assistance platform 102 can include one or more model training procedures 202. The system(s) 100 depicted in FIG. 2 can be similar to, identical to, and/or can form at least a portion of the system(s) depicted in FIGS. 1A and 1B.

In some examples, the model training procedure(s) 202 can be performed prior to the assistance operations, discussed above. The training procedure(s) 202 can transform an ML model 204 into the trained ML model 141. Additionally, the training procedure 202 can use a training data set 205 generated based on a plurality of previous healthcare treatment encounters 206 (e.g., health records, videos, transcripts, and so forth). Each previous healthcare treatment encounter 208 of the plurality of previous healthcare treatment encounters 206 can comprise a data entry associated with various training labels 210 characterizing that healthcare treatment encounter 208. The training labels 210 can be generated and assigned to the previous healthcare treatment encounter 208 manually via a supervised learning process, and/or a self-supervised learning (SSL) training model can be used to generate some or all of the training labels 210, or both. Furthermore, the training data set 205 can undergo an anonymization process using the data anonymizer 129, to remove the personally identifiable information, as discussed above.

For example, the training labels 210 can include one or more context factor label(s) 212 which can indicate a treatment facility type, a treatment facility unit, whether a treatment facility is in an urban region or a region, and/or other data related to the context of the previous healthcare treatment encounter 208 (e.g., a timestamp, a duration, applicable regulations, etc.). Additionally, the training labels 210 can include one or more patient factor label(s) 214 characterizing various features of a patient 216 receiving treatment during the previous healthcare treatment encounter 208. For instance, the patient factor label(s) 214 can include any combination of patient height, patient weight, patient age, patient gender, patient-provided symptoms, vital signs, patient test results, patient risk factors, or other data characterizing the patient 216. Furthermore, the training label(s) 210 can include one or more injury/illness factor label(s) 218 indicating an injury/illness type, an injury/illness severity, an injury/illness duration the patient-provided symptoms, the vital signs, the patient test results, and/or any combinations thereof, and so forth. The training label(s) 210 can also include one or more healthcare personnel factor label(s) 220 providing data related to characteristics of a healthcare personnel 222 providing treatment during the healthcare treatment encounter 208. The healthcare personnel factor label(s) 220 can indicate one or more of an occupation, a specialty, an experience level, an age, a gender, a medical school, an undergraduate degree, and/or any combination thereof.

Additionally, in some examples, the training label(s) 210 can include one or more treatment action label(s) 224 indicating treatment actions taken by the healthcare personnel 222 during the previous healthcare treatment encounter 208, such as prescribing a particular medication, a particular medication dosage, a change to a medication dosage, questions asked, a blood test, an imaging procedure, a physical therapy instruction, examination actions, specialists consulted, a patient monitoring instruction or duration, and/or any combination thereof. Furthermore, the training label(s) 210 can include one or more patient outcome label(s) 226 indicating various aspects of the patient outcome from the previous healthcare treatment encounter 208. For instance, the patient outcome label(s) 208 can indicate a time frame for healing and/or recovery from the injury/illness, any complications, whether mobility was regained and/or how long it took to regain mobility, whether the patient 216 returned to work and/or how long it took to return to work, any operations resulting from the previous healthcare treatment encounter 208, any infections, and/or any combination thereof. It is to be understood that the training label(s) 210 can include any combination of the examples disclosed herein, and any of these example training label(s) 210 can be combined with any other example training label(s) 210. In some instances, the training label(s) 210 can be assigned using various image recognition engines and/or audio-to-text engines to analyze data representing the previous healthcare treatment encounters 208. Moreover, as discussed in greater detail below, the system 100 can undergo multiple training procedures 202 to generate different iterations of the trained ML model 141.

In some examples, the training procedure 202 can include execution of training classification algorithms, transformers, test algorithm, validation algorithms, and so forth. For instance the ML model 204 can include combinations of supervised, unsupervised, and/or reinforcement models interacting together to calculate the statistical likelihood value 144 of the treatment action(s) 162. For instance, the ML model 204 and/or the trained ML model 141 can include classification and/or regression algorithms, such as a linear and/or logistic regression, a naïve bayes classifier, a neural network, a random forest, a decision tree, a support vector machine, hierarchical or probabilistic clustering, a k-nearest neighbor cluster algorithm, a principle component analysis for dimensional reductions, Q-learning, a Boltzmann machine, and/or combinations thereof.

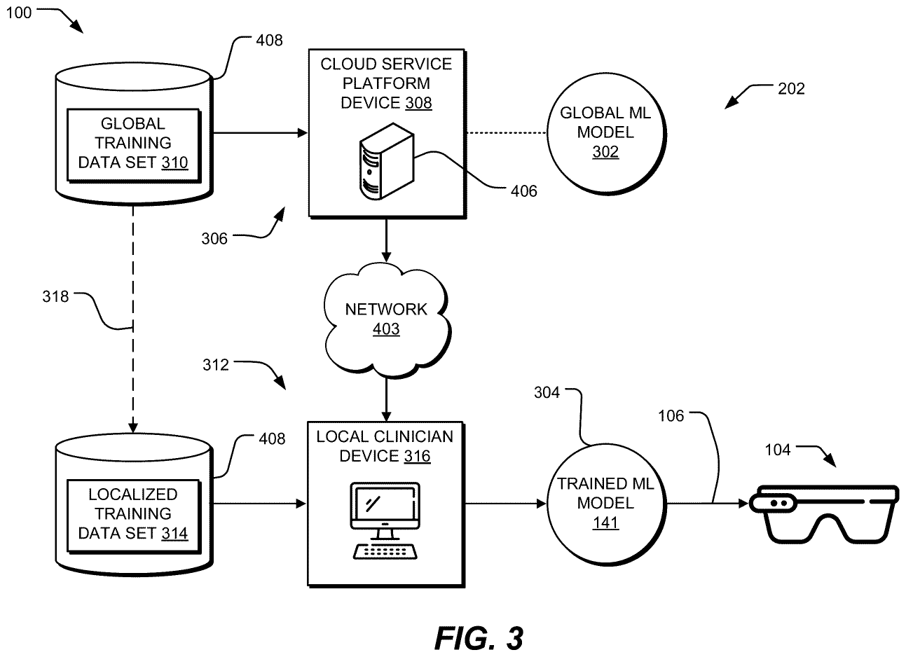

FIG. 3 illustrates an example system 100 for implementing the healthcare assistance platform 102 on a wearable computing device 104. As depicted in FIG. 3, the healthcare assistance platform 102 can include model training procedures 202 for creating a global ML model 302 and a localized ML model 304. The system(s) 100 depicted in FIG. 3 can be similar to, identical to, and/or can form at least a portion of the system(s) depicted in FIGS. 1A and 1B.

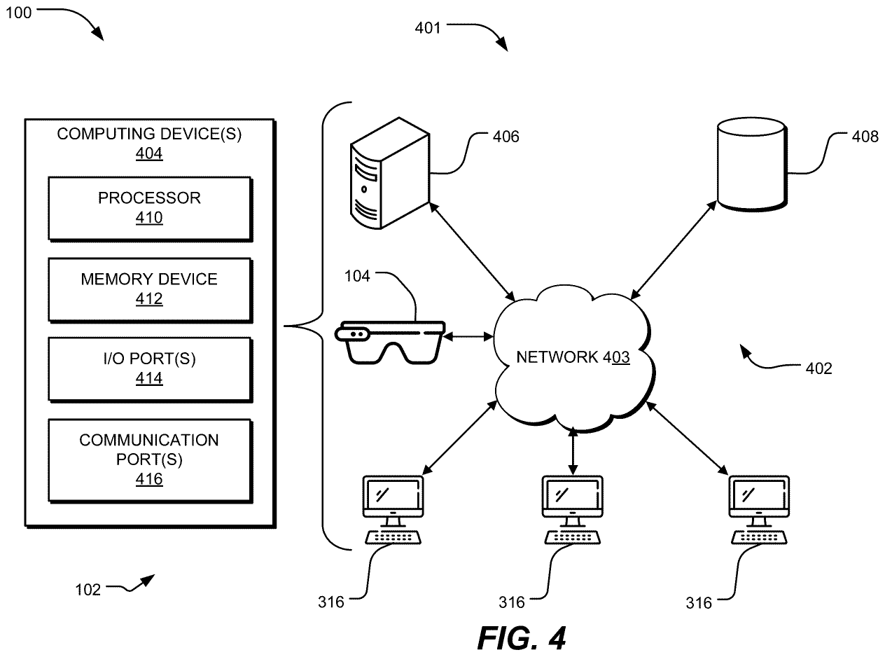

In some examples, the system 100 can include performing a first training procedure 306 to create the global ML model 302. The first training procedure 306 can occur at a cloud service platform device 308 (e.g., as shown in FIG. 4, a server 406 or other type of computing device 404) which hosts the global ML model 126, a global training data set 310, and/or various training algorithms, such as the training algorithms of the training procedure 202, discussed above regarding FIG. 2. The global training data set 310 (e.g., stored in one or more database(s) 408) can include the data related to the plurality of previous healthcare encounters 206 aggregated from a plurality of different healthcare treatment facilities, and can include any of the training labels 210. The trained ML model 141 discussed herein can be the global ML model 302.;

In some instances, the system 100 can include performing a second training procedure 312 to create the localized ML model 304, which can result in the trained ML model 141. The second training procedure 312 can use a localized training data set 314. The localized training data set 314 (e.g., stored in the one or more database(s) 408) can include the training data 210 related to the previous healthcare encounter(s) 208 corresponding to a particular healthcare facility. For instance, the previous healthcare encounter(s) 208 of the localized training data set 314 can have occurred at the particular healthcare facility. Additionally or alternatively, the localized training data set 314 can be particularized to the particular healthcare facility (e.g., or group of healthcare facilities sharing common operating entities) in other ways, such as focusing on a particular specialty, type of injury, and/or type of illness more prevalent at that particular healthcare facility. Furthermore, the localized training data set 314 can include different weights relative to the global training data set 310. For example, previous healthcare encounter(s) 208 occurring at the particular healthcare facility can be weighed higher than previous healthcare encounter(s) 208 occurring at other healthcare facilities. In this way, the localized training data set 314 can be used to generate the localized ML model 304 using less data than that used for generating the global ML model 302. In other words, the first training procedure 306 and the second training procedure 312 can be combined by weighing particular data entries of the globalized training data set 310 (e.g., which are more relevant to the particular healthcare facility) higher than other data entries, converting the global training data set 310 into the localized training data set 314 (e.g., as part of a data set localization procedure 318).

In some scenarios, the second training procedure 312 can occur at a local clinician device 316 or other device associated with the particular healthcare facility (e.g., a mobile device or table of the healthcare personnel 131). For instance, the global ML model 302 can be sent from the cloud service platform device 308 to the local clinician device 316. Some or all of the localized training data set 314 can also be sent from the cloud service platform device 308 to the local clinician device 316, and/or some or all of the localized training data set 314 can come from other sources, such as a local database 408 at the particular healthcare facility. Additionally or alternatively, the operations of the second training procedure 312 to generate the localized ML model 304 can occur at the cloud service platform device 308. The localized ML model 304 can then be sent to the local clinician device 308. Additionally or alternatively, the localized ML model 304 can be stored at the cloud service platform device 308 and can accessed by the local clinician device 316 via a web portal and/or a network (e.g., network 403, as shown in FIG. 4).

FIG. 4 illustrates an example system 100 for implementing a cloud service platform 401 for providing at least some of the features of the healthcare assistance platform 102 using a networked architecture 402. The system(s) 100 depicted in FIG. 4 can be similar to, identical to, and/or can form at least a portion of the system(s) 100 depicted in FIGS. 1A and 1B.

In some examples, the system 100 includes one or more computing devices 404 forming the networked architecture 402. The computing device(s) 404 can include the wearable computing device 104, the cloud service platform device 308, the local clinician device 316, and/or other devices associated with personnel using the healthcare assistance platform 102. Moreover, the computing device(s) 404 can include a computer, a personal computer, a desktop computer, a laptop computer, a terminal, a workstation, a cellular or mobile phone, a mobile device, a smart mobile device, a tablet, various wearable devices (e.g., a smart watch, smart glasses, a smart epidermal device, etc.), a multimedia console, a camera, a microphone, a television, an Internet-of-Things (IoT) device, a smart home device, a virtual reality (VR) device, an augmented reality (AR) device, a vehicle device, combinations thereof, and/or the like. It will be appreciated that specific implementations of these devices may be of differing possible specific computing architectures.

In some cases, the networked architecture 402 can include a plurality of local clinician device(s) 316 operating with their localized ML models 304. These localized ML models 304 can operate entirely separately from each other, such that the localized ML models 304 continue to improve towards a customized focus on the particular needs unique to the particular healthcare treatment facilities using the local clinician devices 316. Additionally or alternatively, the localized ML models 304 can be used as validation models between the different local clinician device(s) 316. An API running the training procedure 202 can be used to teach the localized ML models 304 to learn from each other via validation and reinforcement protocols.

In some examples, the computing device(s) 404 discussed herein can communicate via one or more network(s) 403 including any type of network 403, such as the Internet, an intranet, a Virtual Private Network (VPN), a Voice over Internet Protocol (VoIP) network, a wireless network (e.g., Bluetooth®), a cellular network (e.g., 4G, 5G, LTE, etc.), a satellite network, combinations thereof, etc. The network 403 can include communications network(s) with numerous components such as, but not limited to gateways routers, server(s) 406, and registrars, which enable communication across the network 403. In one implementation, the communications network(s) includes multiple ingress/egress routers, which may have one or more ports, in communication with the network 403. Additionally, or alternatively, the computing device(s) 404 and/or server(s) 406 can access and be accessed by the network 403 via another type of communications network, which may be a public switched telephone network (PSTN) operated by a local exchange carrier (LEC).

In some instances, at least one server 406 can host a website or application of the cloud service platform 401, such as a web client application, a download link, and/or other component to provide access to the healthcare assistance platform 102 (e.g., the trained ML model 141, the global ML model 302, the localized ML model, the data pre-processing model(s) 119, the scenario prediction model 132, combinations thereof, and the like). The computing device(s) 404 may visit the hosted website to access the cloud service platform 401 and/or to send inputs (e.g., the input data 114) from an extension, plug-in, and/or application of the healthcare assistance platform 102 to a cloud service platform device 308 (e.g., the server 406). To perform the operations disclosed herein, the server(s) 406 and/or a client device (e.g., the wearable computing device 104 and/or the local clinician device 316) can access (e.g., read and/or write) one or more database(s) 408, for instance, storing the input data, output data, and operational instructions of the healthcare assistance platform 102.

In some instances, a website or application of the system 100 can receive the inputs and can analyze the inputs to generate outputs for the healthcare assistance platform 102 which can be stored at the database(s) 408 (e.g., the statistical likelihood value(s) 144 of the patient outcome(s) 146, the treatment action(s) 162, the healthcare personnel assessment(s) 112, and/or combinations thereof). Furthermore, the server 406 may be a single server, a plurality of servers with each such server being a physical server or a virtual machine, or a collection of both physical servers and virtual machines. In another implementation, the cloud service platform 401 hosts components of the healthcare assistance platform 102 on separate servers 406 operating in parallel. The server(s) 406 may represent an instance among large instances of application servers in a cloud computing environment, a data center, or other computing environment.

Furthermore, the computing device 404 can be a computing system capable of executing a computer program product to execute a computer process of the healthcare assistance platform 102. Data and program files may be input to the computing device 404, which reads the files and executes the programs therein. Some of the elements of the computing device 404 can include one or more hardware processors 410, one or more memory devices 412, and/or one or more ports, such as input/output (IO) port(s) 414 and communication port(s) 416. Various elements of the computing device 404 may communicate with one another by way of the communication port(s) 416 and/or one or more communication buses, point-to-point communication paths, or other communication means.

The processor 410 can include, for example, a central processing unit (CPU), a microprocessor, a microcontroller, a digital signal processor (DSP), a graphics processing unit (GPU), and/or one or more internal levels of cache. There may be one or more processors 410, such that the processor 410 comprises a single central-processing unit, or a plurality of processing units capable of executing instructions and performing operations in parallel with each other, referred to as a parallel processing environment (e.g., across multiple GPUs).

The computing device 404 may be a single computer, a plurality of computers (e.g., a distributed computer), or another type of computer, such as one or more external computers made available via the cloud computing architecture. The presently described technology is optionally implemented in software stored on a data storage device(s) such as the memory device(s) 412 (e.g., locally stored at the computing device 404), and/or communicated via one or more of the ports 414 or 416 to the healthcare assistance platform 102, thereby transforming the computing device 404 into a special purpose machine for providing the features of the healthcare assistance platform 102.

The one or more memory device(s) 412 may include any non-volatile data storage device capable of storing data generated or employed within the computing device 404, such as computer executable instructions for performing a computer process, which may include instructions of both application programs and an operating system (OS) that manages the various components of the computing device 404. The memory device(s) 412 may include, without limitation, magnetic disk drives, optical disk drives, solid state drives (SSDs), flash drives, and the like. The memory device(s) 412 may include removable data storage media, non-removable data storage media, and/or external storage devices made available via a wired or wireless network with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The one or more memory device(s) 412 may include volatile memory (e.g., dynamic random-access memory (DRAM), static random-access memory (SRAM), etc.) and/or non-volatile memory (e.g., read-only memory (ROM), flash memory, etc.).

Computer program products containing mechanisms to effectuate the systems and methods in accordance with the presently described technology may reside in the memory device(s) 412 which may be referred to as machine-readable media. It will be appreciated that machine-readable media may include tangible non-transitory medium capable of storing or encoding instructions to perform operations of the system 100 disclosed herein The machine-readable media can store computer-readable instructions for execution by a machine, and/or can be capable of storing or encoding data structures and/or modules utilized by or associated with such instructions.

In some implementations, the computing device 404 can include one or more ports, such as the I/O port 414 and the communication port 416, for communicating with other computing, network, or devices. It will be appreciated that the I/O port 414 and the communication port 416 may be combined or separate and that more or fewer ports may be included in the computing device 404.

The I/O port 414 may be connected to an I/O device, or other device, by which information is input to or output from the computing device 404. For instance, input devices can convert a human-generated signal, such as, human voice, physical movement, physical touch or pressure, and/or the like, into electrical signals as input data into the computing device 404 via the I/O port 414. Similarly, output devices may convert electrical signals received from the computing device 404 via the I/O port 414 into signals that may be sensed as output by a human, such as sound, light, and/or touch. The input device may be an alphanumeric input device, including alphanumeric and other keys for communicating information and/or command selections to the processor 410 via the I/O port 414. The input device may be another type of user input device including, but not limited to: direction and selection control devices, such as a mouse, a trackball, cursor direction keys, a joystick, and/or a wheel; one or more sensors, such as a camera, a microphone, a positional sensor, an orientation sensor, an inertial sensor, an accelerometer; and/or a touch-sensitive display screen (“touchscreen”). The output devices may include, without limitation, a display, a touchscreen, a speaker, a tactile or haptic output device, and/or the like. In some implementations, the input device and the output device may be the same device, for example, in the case of a touchscreen.

In one implementation, the communication port 416 is connected to the network 403, and the computing device 404 may receive network data useful in executing the methods and systems set out herein as well as transmitting information and network configuration changes determined thereby. Stated differently, the communication port 416 can connect the computing device 404 to one or more communication interface devices configured to transmit and/or receive information between the computing device 404 and other devices by way of one or more wired or wireless communication network connections. Examples of such networks connections include, without limitation, Universal Serial Bus (USB), Ethernet, Wi-Fi, Bluetooth®, Near Field Communication (NFC), or any other network connection interface of the network 403. For instance, one or more such communication interface devices may be utilized via the communication port 416 to communicate with one or more other machines, either directly over a point-to-point communication path, over a wide area network (WAN) (e.g., the Internet), over a local area network (LAN), over a cellular network, or over another communication means. Further, the communication port 416 may communicate with an antenna or other link for electromagnetic signal transmission and/or reception.

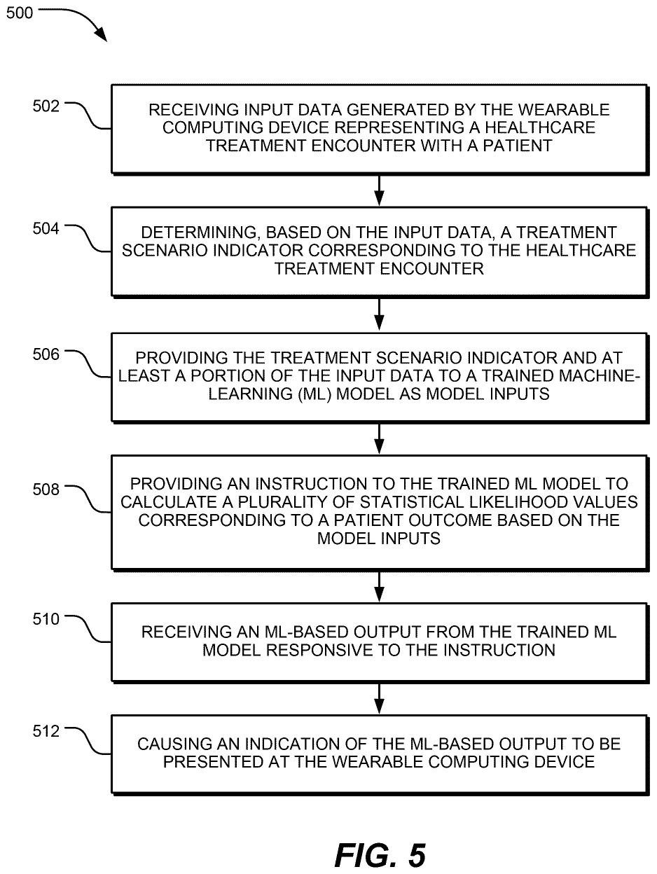

FIG. 5 depicts an example method 500 of providing a healthcare treatment assistance tool including a wearable computing device 104, which can be performed by any of the system(s) 100 disclosed in FIGS. 1-4.

In some instances, at operation 502, the method 500 can receive input data generated by the wearable computing device representing a healthcare treatment encounter with a patient. At operation 504, the method 500 can determine, based on the input data, a treatment scenario indicator corresponding to the healthcare treatment encounter. At operation 406, the method 500 can provide the treatment scenario indicator and at least a portion of the input data to a trained ML model as model inputs. At operation 508, the method 500 can provide an instruction to the trained ML model to calculate a plurality of statistical likelihood values corresponding to a patient outcome based on the model inputs. At operation 510, the method 500 can receive an ML-based output from the trained ML model responsive to the instruction. At operation 512, the method 500 can cause an indication of the ML-based output to be presented at the wearable computing device.

It is to be understood that the specific order or hierarchy of steps in the methods depicted throughout this disclosure are instances of example approaches and can be rearranged while remaining within the disclosed subject matter. For instance, any of the operations discussed throughout this disclosure may be omitted, repeated, performed in parallel, performed in a different order, and/or combined with any other of the operations discussed throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. Any feature from any example disclosed herein can be combined with any other feature. These and other variations, modifications, additions, and improvements may fall within the scope of the disclosure as defined in the claims that follow.

What is claimed is:

1. A healthcare training system, the system including:
a wearable computing device;
one or more processors; and
one or more memory devices storing instructions which, when executed by the one or more processors, cause the system to:
generate, at the wearable computing device, input data including audio data and imagery data representing a healthcare treatment encounter with a patient;
determine, based on the input data, a treatment scenario indicator corresponding to the healthcare treatment encounter;
provide the treatment scenario indicator and at least a portion of the input data to a trained machine-learning (ML) model as model inputs;
instruct the trained ML model to calculate a statistical likelihood value corresponding to a patient outcome based on the model inputs;
receive an ML-based output from the trained ML model responsive to the instructing of the trained ML model to calculate the statistical likelihood value; and
present an indication of the ML-based output at the wearable computing device.

2. The system of claim 1, wherein,
the determining of the treatment scenario indicator includes:
providing the imagery data to an image recognition model as a first model input,
receiving a predicted environment data output from the image recognition model,
providing the audio data to a audio-to-text converter model as a second model input,
receiving a text data output from the audio-to-text converter model, and
providing the predicted environment data output and the text data output to a scenario prediction model as third model inputs.

3. The system of claim 1, wherein,
the treatment scenario indicator includes one or more treatment context factors including at least one of:
a treatment facility type,
a treatment facility unit, or
a determination of a rural treatment region or an urban treatment region.

4. The system of claim 3, wherein,
the treatment scenario indicator includes one or more patient factors including at least one of:
an injury type,
an injury severity,
one or more patient-provided symptoms,
one or more vital signs,
one or more patient test results, or one or more patient risk factors.

5. The system of claim 4, wherein,
the treatment scenario indicator includes one or more healthcare personnel factors including at least one of:
a healthcare personnel occupation,
a healthcare personnel specialty,
a healthcare personnel experience level, or
a prior treatment activity.

6. The system of claim 1, wherein,
the instructions, when executed by the one or more processors, cause the system to:
instruct the trained ML model to determine one or more treatment actions that increase the statistical likelihood value corresponding to the patient outcome,
receive a treatment action output from the trained ML model, and
present an indication of the treatment action output at a display of the wearable computing device.

7. The system of claim 1, wherein,
the instructions, when executed by the one or more processors, cause the system to:
provide a training data set to an ML model resulting in the trained ML model, the training data set including a plurality of previous healthcare encounters associated with training labels.

8. The system of claim 7, wherein,
the training labels include:
context factor labels,
patient factor labels,
healthcare personnel factor labels,
treatment action labels, and
patient outcome labels.

9. The system of claim 7, wherein,
the ML model includes a previously-trained global ML model provided at a server of a cloud service, and
the plurality of previous healthcare encounters correspond to a particular healthcare facility implementing an ML-based healthcare assistance platform from the cloud service such that the trained ML model is a localized trained ML model corresponding to the particular healthcare facility.

10. The system of claim 9, wherein,
the input data is first input data,
the ML-based output is a first ML-based output, and
the instructions, when executed by the one or more processors, cause the system to:
provide second input data representing a plurality of healthcare treatment encounters, particular to a wearer of the wearable computing device, to the trained ML model,
receive, from the trained ML model responsive to the second input data, a second ML-based output representing a healthcare personnel assessment, and
causing an indication of the second ML-based output to be presented at the wearable computing device.

11. The system of claim 10, wherein,
the healthcare personnel assessment includes a readiness level for a particular procedure.

12. A healthcare training system, the system including:
one or more processors; and
one or more memory devices storing instructions which, when executed by the one or more processors, cause the system to:
receive input data generated by a wearable computing device, the input data including audio data and imagery data representing a healthcare treatment encounter with a patient;
determine, based on the input data, a treatment scenario indicator corresponding to the healthcare treatment encounter;
provide the treatment scenario indicator and at least a portion of the input data to a trained machine-learning (ML) model as model inputs;
instruct the trained ML model to calculate a statistical likelihood value corresponding to a patient outcome based on the model inputs;
receive a first ML-based output from the trained ML model responsive to the instructing of the trained ML model to calculate the statistical likelihood value;
instruct the trained ML model to determine one or more treatment actions that increase the statistical likelihood value corresponding to the patient outcome;
receive a second ML-based output from the trained ML model responsive to the instruction of the trained ML model to determine the one or more treatment actions; and
cause an indication of the second ML-based output to be presented at the wearable computing device.

13. The system of claim 12, wherein,
the patient outcome is selected, based at least in part on the treatment scenario indicator, from a group of patient outcomes including one or more of:
a likelihood of complications,
a likelihood of regaining mobility,
a likelihood of returning to work within a predetermined time frame,
a likelihood of requiring an operation, or a likelihood of infection; and
an outcome pathway timeline associated with the patient is adjusted based at least in part on the patient outcome.

14. The system of claim 13, wherein,
the treatment scenario indicator includes a treatment context factor, a patient factor, and a healthcare personnel factor.

15. The system of claim 14, wherein,
the causing of the indication of the second ML-based output to be presented at the wearable computing device includes causing a visualization of the one or more treatment actions to be overlayed on an a live-feed of a real-world image of the healthcare treatment encounter.

16. A method of healthcare treatment assistance with a wearable computing device, the method comprising:
receiving input data generated by the wearable computing device representing a healthcare treatment encounter with a patient;
determining, based on the input data, a treatment scenario indicator corresponding to the healthcare treatment encounter;
providing the treatment scenario indicator and at least a portion of the input data to a trained machine-learning (ML) model as model inputs;
providing a first instruction to the trained ML model to calculate a plurality of statistical likelihood values corresponding to a patient outcome based on the model inputs;
receiving a first ML-based output from the trained ML model responsive to the first instruction; and
causing an indication of the first ML-based output to be presented at the wearable computing device.

17. The method of claim 16, further comprising:
providing a second instruction to the trained ML model to determine one or more treatment actions that increase the statistical likelihood value corresponding to the patient outcome,
receiving a second ML-based output from the trained ML model responsive to the instruction of the trained ML model to determine the one or more treatment actions, and
causing an indication of the second ML-based output to be presented at the wearable computing device.

18. The method of claim 17, wherein,
the input data includes audio data and imagery data, and
the determining of the treatment scenario indicator includes:
providing the imagery data to an image recognition model as a first model input,
receiving a predicted environment data output from the image recognition model,
providing the audio data to a audio-to-text converter model as a second model input,
receiving a text data output from the audio-to-text converter model, and
providing the predicted environment data output and the text data output to a scenario prediction model as third model inputs.

19. The method of claim 16, further comprising:
establishing a wireless network connection between the wearable computing device and a local clinician device associated with a treatment facility of a wearer of the wearable computing device, the trained ML model being stored at the local clinician device.

20. The method of claim 19, further comprising:
training a global ML model, at a cloud service device, with a first training data set resulting in a trained global ML model at the cloud service device,
sending the global ML model to the local clinician device resulting in a local ML model, and
training the local ML model with a second training data set, particularized for the treatment facility, resulting in the trained ML model as a localized trained ML model.
